(19) United States
(12) Patent Application Publication
(10) Pub. No.: US 2026/0237026 A1
(43) Pub. Date: Aug. 13, 2026

(54) ENDOSCOPIC IMAGE ENHANCEMENT METHOD AND DEVICE

(71) Applicant: SEOUL NATIONAL UNIVERSITY R&DB FOUNDATION, Seoul (KR)

(72) Inventors: Sungwan KIM, Seoul (KR); Dan YOON, Seoul (KR); Byeongsoo KIM, Seoul (KR); Woosang CHO, Seoul (KR); Hyoun-Joong KONG, Seoul (KR); Minwoo CHO, Seoul (KR)

(21) Appl. No.: 19/158,852

(22) PCT Filed: Mar. 28, 2024
(86) PCT No.: PCT/KR2024/003997

§ 371 Date: Aug. 21, 2025

(30) Foreign Application Priority Data
Apr. 10, 2023 (KR) 10-2023-0047025

(51) Int. Cl. G06T 5/60; G06T 11/10

(52) U.S. Cl. CPC G06T 5/60; G06T 11/10; G06T 2207/10068; G06T 2207/20081; G06T 2207/20084

(57) ABSTRACT

Described are an endoscopic image enhancement method and device, enabling an enhanced image in which a specific region is highlighted to be generated from a white light image provided by an endoscope. The endoscopic image enhancement method comprises: checking a white light image captured by an endoscope; inputting the white light image into a pre-trained enhanced image generation model; and checking an enhanced endoscopic image corresponding to the white light image, generated by means of the pre-trained enhanced image generation model.

### TECHNICAL FIELD

The present invention relates to an endoscopic image enhancement method and device capable of generating various types of enhanced images from a white light image provided by an endoscopic device.

This work was supported by Korea Medical Device Development Fund funded by the Korea government (Ministry of Science and ICT) (Project unique No.: 1711179421; Project No.: KD000315; R&D project: (Foundation) Inter-Ministry Full Cycle Medical Device Research and Development Project Group; Research Project Title: Development and commercialization of a high-performance smart flexible electronic endoscope system for the digestive system based on artificial intelligence and electrification technology; and Project period: 2023 Jan. 1~2023 Dec. 31).

### BACKGROUND ART

In a medical environment, doctors diagnose lesions by comprehensively checking conditions, colors, shapes, distributions, and patterns of blood vessels, changes in color tone, etc., of a subject through an endoscopic examination. Even if the above-described information is checked, it is difficult to detect subtle changes in lesions at an early stage. In order to address these limitations, endoscopic technologies are being developed to provide various improved feature images through innovative optical technologies using special image processing technologies or specialized filters.

For example, the conventional endoscopic device provides operation modes such as a normal observation mode that irradiates white light onto a subject inside a living body and captures an image of the subject to provide a normal endoscopic image, i.e., a white light image, and a narrowband mode that irradiates a subject with light having a narrower wavelength band than the illumination of the normal observation mode to provide an enhanced image, i.e., an augmented image, in which blood vessels, etc., on a mucosal surface appear clearly.

Therefore, users, i.e., doctors who observe and diagnose patients' lesions through the endoscopic device primarily observe a suspected lesion site from the white light image through the normal observation mode, and perform a diagnosis of a lesion through clearer observation of a suspected area from the enhanced image of the suspected site through the narrowband mode.

However, in order to provide the normal observation mode and the narrowband mode in the conventional endoscopic device, hardware for mode control, for example, hardware such as a filter that may control light wavelengths differently, should be added to the endoscopic device, which causes a problem in which the structure of the endoscopic device becomes complex.

In addition, the conventional endoscopic device provides users with an enhanced image according to a single narrowband mode determined by the manufacturer of the endoscopic device due to hardware characteristic limitations, and thus, has a problem of reduced versatility of the endoscopic device in providing the narrowband mode.

### DISCLOSURE

### Technical Problem

The present invention provides an endoscopic image enhancement method and device capable of generating various types of enhanced images from a white light image provided by an endoscopic device.

### Technical Solution

The method for enhancing endoscopic image according to an embodiment of the present invention, the method comprising: receiving a white light image captured by an endoscopic device; inputting the white light image into a pre-trained enhanced image generation model; and generating an endoscopic enhanced image by enhancing the white light image using the pre-trained enhanced image generation model.

Wherein the enhanced image generation model may include a generator and a discriminator, wherein the generator may be trained to generate the endoscopic enhanced image corresponding to the white light image, and wherein the discriminator may receive label data including the endoscopic enhanced image and information on authenticity of the endoscopic enhanced image from the generator, and may be trained to output discriminator information that discriminates the authenticity of the endoscopic enhanced image.

Wherein the discriminator may compare the information on the authenticity of the endoscopic enhanced image included in the label data with a result value of discriminating the authenticity of the endoscopic enhanced image to generate a discriminator loss value, and wherein the generator may generate the endoscopic enhanced image from the white light image by reflecting the discriminator loss value.

Wherein the enhanced image generation model may include a plurality of generators each trained to output a different form of endoscopic enhanced image for the white light image, and wherein the generating the endoscopic enhanced image may include: selecting one or more trained generators from among the plurality of generators; and generating the endoscopic enhanced image corresponding to the white light image using the selected one or more trained generators.

The method may further comprise generating a segmented image including the white light image and the endoscopic enhanced image corresponding to the white light image; and outputting the segmented image via a display.

Wherein the endoscopic enhanced image may be configured to include a color corresponding to at least one light wavelength band of green light and blue light.

A device for enhancing endoscopic image according to an embodiment of the present invention, the device comprising: a memory storing one or more instructions; and a processor executing the one or more instructions stored in the memory, wherein the one or more instructions, when executed by the processor, cause the processor to: receive a white light image captured by an endoscopic device; input the white light image into a pre-trained enhanced image generation model; and generate an endoscopic enhanced image by enhancing the white light image using the pre-trained enhanced image generation model.

Wherein the enhanced image generation model may include a generator and a discriminator, wherein the generator may be trained to generate the endoscopic enhanced image corresponding to the white light image, and wherein the discriminator may receive label data including the endoscopic enhanced image and information on authenticity of the endoscopic enhanced image from the generator, and may be trained to output discriminator information that discriminates the authenticity of the endoscopic enhanced image.

Wherein the discriminator may compare the information on the authenticity of the endoscopic enhanced image included in the label data with a result value of discriminating the authenticity of the endoscopic enhanced image to generate a discriminator loss value, and wherein the generator may generate the endoscopic enhanced image from the white light image by reflecting the discriminator loss value.

Wherein the enhanced image generation model may include a plurality of generators each trained to output a different form of endoscopic enhanced image for the white light image, and wherein the one or more instructions, when executed by the processor, may cause the processor to further: select one or more trained generators from among the plurality of generators; and generate the endoscopic enhanced image corresponding to the white light image using the selected one or more trained generators.

Wherein the one or more instructions, when executed by the processor, may cause the processor to further: generate a segmented image including the white light image and the endoscopic enhanced image corresponding to the white light image; and output the segmented image via a display.

Wherein the one or more instructions, when executed by the processor, may cause the processor to further: generate the endoscopic enhanced image including a color corresponding to at least one light wavelength band of green light and blue light.

### Advantageous Effects

According to the present invention, it is possible to generate and output various types of enhanced images in which specific regions inside the human body are highlighted from a white light image provided by an endoscopic device using a trained neural network model.

Accordingly, according to the present invention, it is possible to generate and provide various types of enhanced images desired by a user from a general white light image without adding separate hardware such as a wavelength filter or a special light source to an endoscopic device.

In addition, according to the present invention, by allowing a white light image and an enhanced image to be displayed together on a single screen to enable a user to meticulously observe a difference between the white light image and the enhanced image, the accuracy of observation and diagnosis of a patient's lesion can be improved.

### DESCRIPTION OF DRAWINGS

FIG. 1 is a diagram illustrating an endoscopic image enhancement device according to an embodiment of the present invention.

FIG. 2 is a diagram conceptually illustrating the function of the image enhancement program of FIG. 1.

FIG. 3 is a diagram illustrating a learning method of an enhanced image generation unit according to one embodiment of the present invention.

FIG. 4 is a diagram illustrating a learning method of an enhanced image generation unit according to another embodiment of the present invention.

FIG. 5 is a diagram illustrating an endoscopic image enhancement method according to an embodiment of the present invention.

### MODE FOR DISCLOSURE

The advantages and features of the embodiments and the methods of accomplishing the embodiments will be clearly understood from the following description taken in conjunction with the accompanying drawings. However, embodiments are not limited to those embodiments described, as embodiments may be implemented in various forms. It should be noted that the present embodiments are provided to make a full disclosure and also to allow those skilled in the art to know the full range of the embodiments. Therefore, the embodiments are to be defined only by the scope of the appended claims.

In describing embodiments of the present invention, if it is considered that a detailed description of a known function or configuration may unnecessarily obscure the gist of the present invention, the detailed description will be omitted. In addition, the terms described below are terms defined in consideration of functions in the embodiments of the present invention, the terms may vary according to the intention or precedent of a technician working in the field, the emergence of new technologies, and the like. Therefore, the terms used in the present disclosure should be defined based on the meaning of the terms and the overall contents of the present disclosure, not just the name of the terms.

Hereinafter, a preferred embodiment of the present invention will be described in detail with reference to the accompanying drawings.

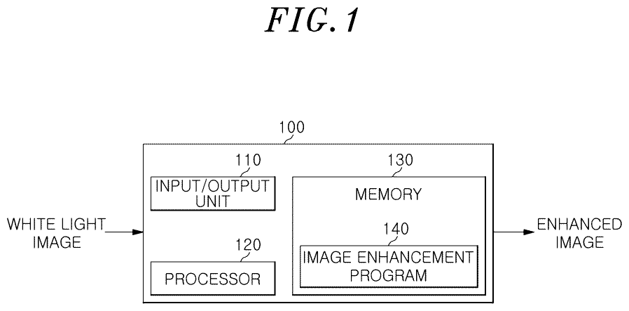

FIG. 1 is a diagram illustrating an endoscopic image enhancement device according to an embodiment of the present invention.

Referring to FIG. 1, an endoscopic image enhancement device 100 of the present embodiment may receive an endoscopic image of an inside of a human body from the outside, for example, an endoscopic device (not illustrated), and generate one or more enhanced images corresponding to the endoscopic image. The endoscopic image enhancement device 100 may output the generated enhanced image to the outside, for example, a display device (not illustrated), and provide the generated enhanced image to a user, such as a doctor who operates the endoscopic device.

The endoscopic image enhancement device 100 may include an input/output unit 110, a processor 120, and a memory 130.

The input/output unit 110 may receive an endoscopic image captured by the endoscopic device, for example, a white light image including a light wavelength band of red (R), green (G), and blue (B). In addition, the input/output unit 110 may output an enhanced image generated in response to the white light image by the processor 120 to be described below to the display device.

Here, the enhanced image may be an image that is enhanced so that a specific region of the white light image is highlighted in comparison with the remaining region. For example, the enhanced image may be an image that is enhanced so that the specific region is highlighted in a predetermined color by including at least one of a green light wavelength band or a blue light wavelength band among multiple light wavelength bands of the white light image. In addition, the enhanced image may be an image that is enhanced so that a contrast of the specific region is highlighted by adjusting a contrast ratio of each part of the white light image to be different.

In addition, according to an embodiment of the present invention, the processor 120 may generate a segmented image including the white light image and the enhanced image, and the input/output unit 110 may output the segmented image to the display device. In this case, the segmented image may be an image that is generated so that the white light image and the enhanced image are each displayed together in a predetermined size on one screen of the display device.

The processor 120 may receive the white light image from the input/output unit 110 and generate the enhanced image from the white light image using an image enhancement program 140 stored in the memory 130 to be described below.

In addition, the processor 120 may output the generated enhanced image to the input/output unit 110 or generate a segmented image including the white light image and the enhanced image and output the generated image to the input/output unit 110.

The memory 130 may store the image enhancement program 140 and information necessary for its execution. The image enhancement program 140 may be software including instructions that may generate the enhanced image in which the specific region is highlighted from the white light image received through the input/output unit 110.

Accordingly, the processor 120 may execute the image enhancement program 140 stored in the memory 130 and generate one or more enhanced images from the white light image received through the input/output unit 110 using the same.

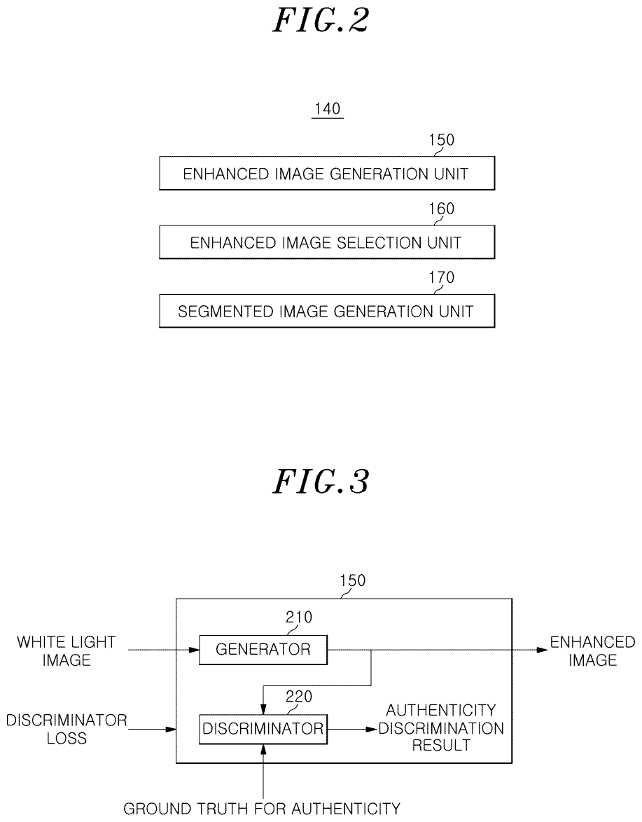

FIG. 2 is a diagram conceptually illustrating the function of the image enhancement program of FIG. 1.

Referring to FIG. 2, the image enhancement program 140 of the present embodiment may include an enhanced image generation unit 150, an enhanced image selection unit 160, and a segmented image generation unit 170.

The enhanced image generation unit 150, the enhanced image selection unit 160, and the segmented image generation unit 170 illustrated in FIG. 2 are conceptually divided to easily describe the function of the image enhancement program 140, and the present invention is not limited thereto.

For example, according to an embodiment of the present invention, the functions of the enhanced image generation unit 150, the enhanced image selection unit 160, and the segmented image generation unit 170 may be merged or separated, and may be implemented as a series of instructions included in one program.

The enhanced image generation unit 150 may generate one or more enhanced images from the white light image. The enhanced image generation unit 150 may include a pre-trained neural network model.

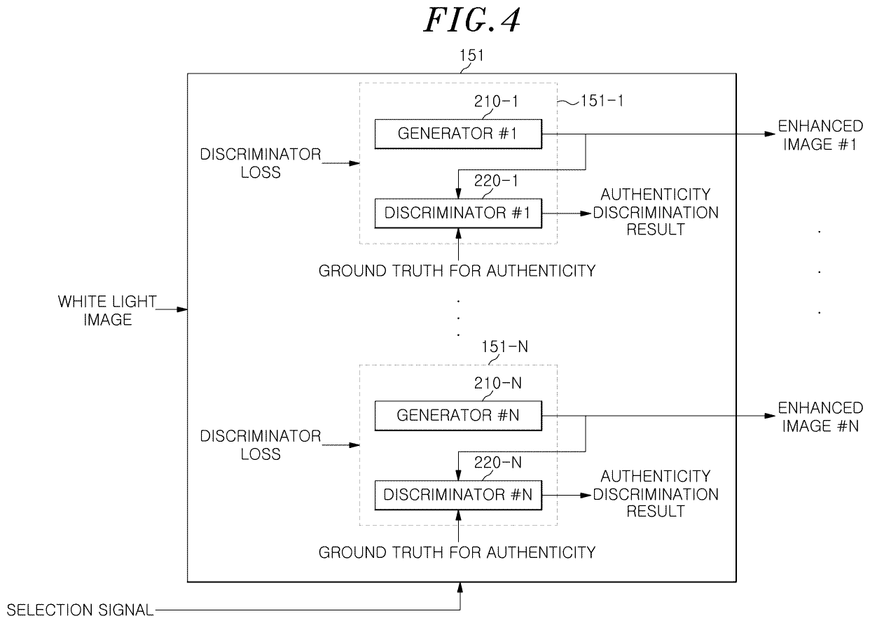

FIG. 3 is a diagram illustrating a learning method of an enhanced image generation unit according to one embodiment of the present invention.

Referring to FIG. 3, the enhanced image generation unit 150 of the present embodiment may include one or more neural network models, such as a generator 210 and a discriminator 220.

The generator 210 may be trained to generate and output the enhanced image corresponding to the white light image when receiving the white light image. The generator 210 may be an auto encoder, and may extract one or more features from the white light image, combine and reconstruct the extracted features, and generate the reconstructed image, i.e., the enhanced image.

Here, the white light image provided to the generator 210 may include images of red, green, and blue light wavelength bands. Accordingly, the generator 210 may generate the enhanced image corresponding to the white light image by combining and reconstructing features of light in various wavelength bands of the input white light image.

The generator 210 may be trained to generate an enhanced image having at least one of various forms, such as narrow band imaging (NBI), blue light imaging (BLI), linked color imaging (LCI), i-scan, and fugi intelligent chromo endoscopy (FICE).

The discriminator 220 may be trained to discriminate and output the authenticity of the enhanced image when receiving the enhanced image generated from the generator 210.

In addition, the discriminator 220 may generate a discriminator loss to further train the generator 210 and the discriminator 220 themselves using the authenticity of the output enhanced image.

To this end, the discriminator 220 may receive a ground truth for the authenticity of the enhanced image, for example, the actual enhanced image along with the enhanced image generated from the generator 210. The discriminator 220 may compare the ground truth for the authenticity with the authenticity actually output, and generate the discriminator loss based on the comparison result.

For example, the discriminator 220 may output a value between 0 and 1 as a result of discriminating the authenticity. The closer the value output from the discriminator 220 is to 0, the more the enhanced image input to the discriminator 220 is discriminated to be fake, and the closer the value output from the discriminator 220 is to 1, the more the enhanced image input to the discriminator 220 is discriminated to be real.

Accordingly, when the discriminator 220 outputs 0.7 as a result of discriminating the authenticity of the input enhanced image, and the ground truth for the authenticity of the enhanced image input as label data to the discriminator 220 is 1, the discriminator 220 may determine 0.3 as a discriminator loss value.

Therefore, the discriminator 220 provides the determined discriminator loss value to the generator 210 and the discriminator 220 themselves, so each of the generator 210 and the discriminator 220 is trained to minimize the discriminator loss value, i.e., the generator 210 is trained to generate the enhanced image and the discriminator 220 is trained to discriminate the authenticity of the enhanced image, which may be repeatedly performed.

Therefore, the enhanced image generation unit 150 of the present embodiment may generate and output the enhanced image corresponding to the white light image provided through the input/output unit 110 using the generator 210 for which the training has been completed.

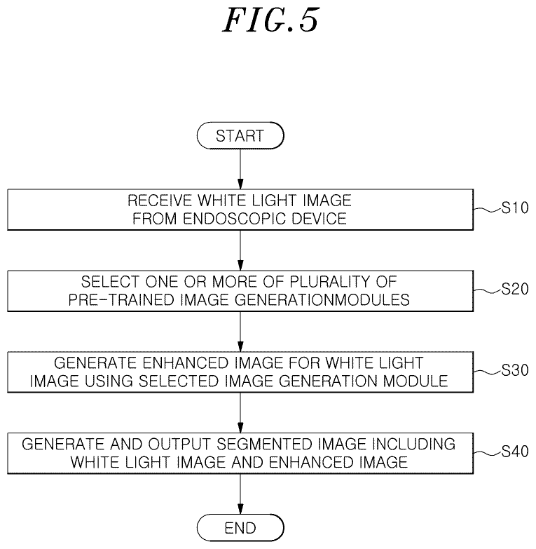

FIG. 4 is a diagram illustrating a learning method of an enhanced image generation unit according to another embodiment of the present invention.

Referring to FIG. 4, an enhanced image generation unit 151 of the present embodiment may include a plurality of image generation modules 151-1 and 151-N. The plurality of image generation modules 151-1 and 151-N may include a first image generation module 151-1 to an Nth image generation module 151-N.

The first image generation module 151-1 may include one or more neural network models, for example, a first generator 210-1 and a first discriminator 220-1. The Nth image generation module 151-N may include an Nth generator 210-N and an Nth discriminator 220-N.

In addition, the first image generation module 151-1 may be trained to generate and output a first enhanced image from the white light image provided through the input/output unit 110. The Nth image generation module 151-N may be trained to generate and output an Nth enhanced image from the white light image provided through the input/output unit 110.

Here, the first enhanced image and the Nth enhanced image may have different forms. For example, the first image generation module 151-1 may generate the first enhanced image having the NBI form from the white light image, and the Nth image generation module 151-N may generate the Nth enhanced image having the i-scan form from the white light image.

Hereinafter, for the convenience of description, the learning method of the first image generation module 151-1 among the plurality of image generation modules 151-1 and 151-N will be described. However, the Nth image generation module 151-N may also perform substantially the same learning method except that the form of the generated enhanced image is different from that of the first image generation module 151-1.

The first generator 210-1 of the first image generation module 151-1 may be trained to generate and output the first enhanced image corresponding to the white light image when receiving the white light image.

The first generator 210-1 may be an autoencoder that extracts one or more features from the white light image and combines and reconstructs the extracted features to generate the reconstructed image, i.e., the first enhanced image.

For example, the white light image may include images of red, green, and blue light wavelength bands. Accordingly, the first generator 210-1 may generate the first enhanced image corresponding to the white light image, i.e., the first enhanced image having the NBI form, by combining and reconstructing features having the remaining light wavelength bands except for the red light wavelength band in the white light image.

The first discriminator 220-1 may be trained to discriminate and output the authenticity of the first enhanced image when receiving the ground truth for the authenticity of the first enhanced image as the label data along with the first enhanced image generated from the first generator 210-1.

In addition, the first discriminator 220-1 may compare the ground truth for the authenticity of the first enhanced image input as the label data with the result of discriminating the authenticity actually output to generate the discriminator loss value, and provide the generated discriminator loss value to the first generator 210-1 and the first discriminator 220-1 themselves. Accordingly, the first generator 210-1 and the first discriminator 220-1 are trained to minimize the discriminator loss value, and for example, the first generator 210-1 is trained to generate the first enhanced image and the first discriminator 220-1 is trained to discriminate the authenticity of the first enhanced image, which may be performed repeatedly.

Accordingly, the enhanced image generation unit 151 of the present embodiment may generate and output the first enhanced image corresponding to the white light image input through the input/output unit 110 using the first image generation module 151-1 for which the training has been completed.

Meanwhile, the enhanced image generation unit 151 of the present embodiment may include the plurality of image generation modules 151-1 and 151-N each trained to generate the enhanced image for the white light image, as described above.

Accordingly, the enhanced image generation unit 151 may generate and output one or more enhanced images through at least one of the plurality of pre-trained image generation modules 151-1 and 151-N based on a selection signal provided by the enhanced image selection unit 160 to be described below.

For example, each of the plurality of image generation modules 151-1 and 151-N may be trained to generate different types of enhanced images from the white light image provided by the endoscopic device. The enhanced image selection unit 160 may generate a selection signal that may select at least one of the plurality of enhanced images based on a user's input and output the selection signal to the enhanced image generation unit 151. Accordingly, the enhanced image generation unit 151 may enable one or more corresponding image generation modules among the plurality of image generation modules 151-1 and 151-N based on the selection signal, and use the enabled image generation module to generate and output the enhanced image corresponding to the selection signal from the white light image, that is, the enhanced image having one of different types of enhanced images.

Referring back to FIG. 2, the enhanced image selection unit 160 may generate the selection signal based on the user's input and output the selection signal to the enhanced image generation unit 150.

For example, the enhanced image generation unit 150 of the present embodiment may include the plurality of image generation modules 151-1 and 151-N as illustrated in FIG. 4, and each of the plurality of image generation modules 151-1 and 151-N may be trained to generate different types of enhanced images.

At least one of the plurality of image generation modules 151-1 and 151-N may be enabled based on the selection signal provided by the enhanced image generation unit 150, and may generate and output the enhanced image from the white light image by the enabled image generation module.

The segmented image generation unit 170 may generate the segmented image that includes the enhanced image output from the enhanced image generation unit 150 and the white light image received through the input/output unit 110. The segmented image may be an image in which the enhanced image and the white light image are each segmented into a predetermined size and displayed together on one screen of the display device.

In this way, the endoscopic image enhancement device 100 of the present embodiment may generate and output an enhanced image for a specific region inside the human body from the white light image provided by the endoscopic device using the trained neural network model.

In addition, the endoscopic image enhancement device 100 of the present embodiment may select one or more of the plurality of neural network models trained to generate different forms of enhanced images, and generate and output the enhanced image of the corresponding form from the white light image.

Therefore, the present invention may easily acquire an enhanced image of a desired form by a user, i.e., a doctor operating the endoscopic device, without adding separate hardware for generating the enhanced image to the endoscopic device.

In addition, the endoscopic image enhancement device 100 of the present embodiment may generate and provide the segmented image so that the white light image and the enhanced image are displayed together on a single screen, thereby allowing the user to meticulously observe the difference between the white light image and the enhanced image to increase the accuracy of observation and diagnosis of the patient's lesion.

FIG. 5 is a diagram illustrating an endoscopic image enhancement method according to an embodiment of the present invention.

Hereinafter, for the convenience of description, an example will be described in which the image enhancement program 140 of the endoscopic image enhancement device 100 includes the enhanced image generation unit 151 illustrated in FIG. 4.

First, according to the user's operation, the endoscopic device may capture the inside of the patient's body and thus output the endoscopic image such as the white light image. The white light image may be received by the input/output unit 110 of the endoscopic image enhancement device 100 (S10).

The processor 120 of the endoscopic image enhancement device 100 may execute the image enhancement program 140 stored in the memory 130 and use the image enhancement program 140 to generate and output one or more enhanced images corresponding to the white light image.

For example, the enhanced image selection unit 160 may generate the selection signal according to the user's input and output the selection signal to the enhanced image generation unit 151. Here, the selection signal may be a signal for selecting at least one of the plurality of enhanced images each having a different form.

The enhanced image generation unit 151 may select one or more image generation modules corresponding to the selection signal among the plurality of pre-trained image generation modules 151-1 and 151-N (S20).

In this case, each of the plurality of image generation modules 151-1 and 151-N may be neural network models trained to generate different types of enhanced images from the white light image, and may be trained as described above with reference to FIG. 4.

Next, the enhanced image generation unit 151 may generate the enhanced image corresponding to the white light image using one or more image generation modules selected from among the plurality of image generation modules 151-1 and 151-N according to the selection signal (S30).

Next, the segmented image generation unit 170 may generate the segmented image that includes the enhanced image generated from the enhanced image generation unit 151 and the white light image provided by the endoscopic device, and may output the segmented image to an external display device and provide the segmented image to the user (S40).

As described above, according to the endoscopic image enhancement method of the present embodiment, it is possible to generate and output various types of enhanced images in which specific regions inside the human body are highlighted from a white light image provided by an endoscopic device using a trained neural network model.

Accordingly, according to the present invention, it is possible to generate and provide various types of enhanced images desired by a user from a general white light image without adding separate hardware such as a wavelength filter or a special light source to an endoscopic device.

In addition, according to the present invention, by allowing a white light image and an enhanced image to be displayed together on a single screen to enable a user to meticulously observe a difference between the white light image and the enhanced image, the accuracy of observation and diagnosis of a patient's lesion can be improved.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable storage medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

1. A method for enhancing endoscopic image to be performed by an endoscopic image enhancement device including a pre-trained enhanced image generation model, the method comprising:
receiving a white light image captured by an endoscopic device;
inputting the white light image into athe pre-trained enhanced image generation model; and
generating an endoscopic enhanced image by enhancing the white light image using the pre-trained enhanced image generation model.

2. The method of claim 1, wherein the enhanced image generation model includes a generator and a discriminator,
wherein the generator is trained to generate the endoscopic enhanced image corresponding to the white light image, and
wherein the discriminator receives label data including the endoscopic enhanced image and information on authenticity of the endoscopic enhanced image from the generator, and is trained to output discriminator information that discriminates the authenticity of the endoscopic enhanced image.

3. The method of claim 2, wherein the discriminator compares the information on the authenticity of the endoscopic enhanced image included in the label data with a result value of discriminating the authenticity of the endoscopic enhanced image to generate a discriminator loss value, and
wherein the generator generates the endoscopic enhanced image from the white light image by reflecting the discriminator loss value.

4. The method of claim 1, wherein the enhanced image generation model includes a plurality of generators each trained to output a different form of endoscopic enhanced image for the white light image, and
wherein the generating the endoscopic enhanced image includes:
selecting one or more trained generators from among the plurality of generators; and
generating the endoscopic enhanced image corresponding to the white light image using the selected one or more trained generators.

5. The method of claim 1, further comprising:
generating a segmented image including the white light image and the endoscopic enhanced image corresponding to the white light image; and
outputting the segmented image via a display.

6. The method of claim 1, wherein the endoscopic enhanced image is configured to include a color corresponding to at least one light wavelength band of green light and blue light.

7. A device for enhancing endoscopic image, the device comprising:
a memory storing one or more instructions and a pre-trained enhanced image generation model; and
a processor executing the one or more instructions stored in the memory,
wherein the one or more instructions, when executed by the processor, cause the processor to:
receive a white light image captured by an endoscopic device;
input the white light image into athe pre-trained enhanced image generation model; and
generate an endoscopic enhanced image by enhancing the white light image using the pre-trained enhanced image generation model.

8. The device of claim 7, wherein the enhanced image generation model includes a generator and a discriminator,
wherein the generator is trained to generate the endoscopic enhanced image corresponding to the white light image, and
wherein the discriminator receives label data including the endoscopic enhanced image and information on authenticity of the endoscopic enhanced image from the generator, and is trained to output discriminator information that discriminates the authenticity of the endoscopic enhanced image.

9. The device of claim 8, wherein the discriminator compares the information on the authenticity of the endoscopic enhanced image included in the label data with a result value of discriminating the authenticity of the endoscopic enhanced image to generate a discriminator loss value, and
wherein the generator generates the endoscopic enhanced image from the white light image by reflecting the discriminator loss value.

10. The device of claim 7, wherein the enhanced image generation model includes a plurality of generators each trained to output a different form of endoscopic enhanced image for the white light image, and
wherein the one or more instructions, when executed by the processor, cause the processor to further:
select one or more trained generators from among the plurality of generators; and
generate the endoscopic enhanced image corresponding to the white light image using the selected one or more trained generators.

11. The device of claim 7, wherein the one or more instructions, when executed by the processor, cause the processor to further:
generate a segmented image including the white light image and the endoscopic enhanced image corresponding to the white light image; and
output the segmented image via a display.

12. The device of claim 7, wherein the one or more instructions, when executed by the processor, cause the processor to further:
generate the endoscopic enhanced image including a color corresponding to at least one light wavelength band of green light and blue light.

13. A non-transitory computer-readable storage medium storing a computer program, wherein the computer program includes one or more instructions to perform a method for enhancing endoscopic image, the method comprising:
receiving a white light image captured by an endoscopic device;
inputting the white light image into a pre-trained enhanced image generation model; and
generating an endoscopic enhanced image by enhancing the white light image using the pre-trained enhanced image generation model.

14. (canceled)
